(19) United States
(12) Patent Application Publication
(10) Pub. No.: US 2025/0092362 A1
(43) Pub. Date: Mar. 20, 2025

(54) System And Methods For Producing Chimeric Antigen Receptor Cells

(71) Applicant: Terumo BCT, Inc., Lakewood, CO (US)

(72) Inventors: Dalip Sethi, Thornton, CO (US); Mindy M. Miller, Lakewood, CO (US); Dennis J. Hlavinka, Lakewood, CO (US)

(21) Appl. No.: 18/823,038

(22) Filed: Sep. 3, 2024

Related U.S. Application Data
(60) Provisional application No. 63/538,610, filed on Sep. 15, 2023.

(51) Int. Cl. C12N 5/078; B01D 15/34; C12M 1/00; C12N 15/86

(52) U.S. Cl. CPC C12N 5/0634; B01D 15/34; C12M 47/04; C12N 15/86; C12N 2510/00; C12N 2740/15041

(57) ABSTRACT

A method for preparing chimeric antigen receptor cells using a cell expansion system includes introducing target cells to a bioreactor of the cell expansion system, where the introduction of the target cells includes isolating the target cells from a source. The isolation of the target cells from the source includes contacting the source to one or more identifying component and then causing the source to move through a separating column, where the separating column disposed in the cell expansion system in line with the bioreactor. The method further includes introducing viral vectors to the bioreactor, introducing a transduction reagent to the bioreactor, and removing non-cellular material from the bioreactor by causing material in the bioreactor to flow through a bypass loop, where the bypass loop includes one or more size exclusion filters and the non-cellular material includes unused viral vectors and unused identifying components.

### CROSS-REFERENCE TO RELATED APPLICATIONS

This application claims the benefit of U.S. Provisional Application No. 63/538,610 filed on Sep. 15, 2023. The entire disclosure of the above application is incorporated herein by reference.

### FIELD

The present disclosure relates to systems and methods for producing chimeric antigen receptor cells.

### BACKGROUND

This section provides background information related to the present disclosure which is not necessarily prior art.

Natural killer cells are innate lymphoid cells that naturally attack certain cells. T-cells are white blood cells that play a central role in adaptive immune responses. Natural killer cells and T-cells are each interesting candidates for various cell therapies and treatments of various malignant diseases, where the therapies and treatments focus on harvesting and supporting the power of the existing immune system. For example, natural killer cells and T-cells have recently been used as cell types for engineered chimeric antigen receptor immunotherapies. Despite their promise, widespread clinical success of natural killer cell therapies and/or treatments and/or T-cells therapies and/or treatments have often been limited because of challenges in easily and efficiently manufacturing large doses of the chimeric antigen receptor cells. Current methods for natural killer cell expansion and/or T-cell cell expansion are often flask based, which can be time consuming, as well as expensive, and have relatively low success rates. It would be desirable to develop improved methods for natural killer cell expansion and/or T-cell expansion and/or other similar cell expansion that have improved success rates and are also less time consuming and expensive.

### SUMMARY

This section provides a general summary of the disclosure, and is not a comprehensive disclosure of its full scope or all of its features.

In various aspects, the present disclosure provides a method for preparing chimeric antigen receptor cells using a cell expansion system.

In at least one example embodiment, the method may include contacting target cells and viral vectors within a bioreactor of the cell expansion system.

In at least one example embodiment, the method may include introducing the target cells to the bioreactor.

In at least one example embodiment, the method may include isolating the target cells from a source, where the source includes the target cells and a non-target material.

In at least one example embodiment, the isolation of the target cells from the source may include contacting the source to one or more identifying components and causing the source including the one or more identifying components to move through a separating column, where the separating column is disposed in the cell expansion system in line with the bioreactor and is configured to allow passage of the target cells and to either hold the non-target material or to direct the non-target material to a holding container of the cell expansion system.

In at least one example embodiment, the one or more identifying components may include magnetically conjugated antibodies selected to associate with the non-target material and non-conjugated antibodies selected to associate with the target cells.

In at least one example embodiment, the method may include identifying the magnetically conjugated antibodies and identifying the non-conjugated antibodies.

In at least one example embodiment, the one or more identifying components may include stimulation beads selected to associate with the target material, magnetic antibody-conjugated beads selected to associate with the target cells, or a combination thereof.

In at least one example embodiment, the method may include identifying the stimulation beads and identifying the magnetic antibody-conjugated beads.

In at least one example embodiment, the separating column may include a magnetic column.

In at least one example embodiment, the method may include introducing the viral vectors to the bioreactor.

In at least one example embodiment, the method may include contacting the target cells and viral vectors with a transduction reagent within the bioreactor.

In at least one example embodiment, the method may include introducing the transduction reagent to the bioreactor.

In at least one example embodiment, the introduction of the transduction reagent may include coating on one or more surfaces of the bioreactor with the transduction reagent.

In at least one example embodiment, the introduction of the transduction reagent may include placing the transduction reagent in suspension with the target cells and the viral vectors.

In at least one example embodiment, the contacting may include maintaining the target cells, the viral vectors, and the transduction reagent within the bioreactor using counterflow containment.

In at least one example embodiment, the method may include removing non-cellular material from the bioreactor, where the non-cellular material includes, for example, unused viral vectors and unused identifying components.

In at least one example embodiment, the removal of the non-cellular material from the bioreactor may include causing material in the bioreactor to flow through a bypass loop, where the bypass loop includes one or more size exclusion filters.

In at least one example embodiment, the removal of the non-cellular material from the bioreactor may include reversing flow and moving a clean media through the bypass loop.

In various aspects, the present disclosure provides another method for preparing chimeric antigen receptor cells using a cell expansion system.

In at least one example embodiment, the method may include introducing target cells to a bioreactor of the cell expansion system, where the introduction of the target cells includes isolating the target cells from a source that includes the target cells and a non-target material. The isolation of the target cells from the source may include contacting the source to one or more identifying component and causing the source including the one or more identifying components to move through a separating column, where the separating column disposed in the cell expansion system in line with the bioreactor and is configured to allow passage of the target cells and to either hold the non-target material or to direct the non-target material to a holding container of the cell expansion system. The method may further include introducing viral vectors to the bioreactor to associate with the target cells, introducing a transduction reagent to the bioreactor to associate with the target cells and the transduction reagent, and removing non-cellular material from the bioreactor by causing material in the bioreactor to flow through a bypass loop, where the bypass loop includes one or more size exclusion filters and the non-cellular material includes unused viral vectors and unused identifying components.

In at least one example embodiment, the method may include maintaining the target cells, the viral vectors, and the transduction reagent within the bioreactor using counterflow containment.

In at least one example embodiment, the introduction of the transduction reagent may include coating on one or more surfaces of the bioreactor with the transduction reagent.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an illustration of an example cell expansion system having a bioreactor in accordance with at least one example embodiment;

FIG. 2 is an illustration of an example bioreactor that shows circulation paths through the bioreactor, and which may be incorporated into cell expansion systems, like the cell expansion system illustrated in FIG. 1, in accordance with at least one example embodiment;

FIG. 3 is an illustration of an example rocking device configured to move a bioreactor, like the bioreactor illustrated in FIG. 2, in accordance with at least one example embodiment;

FIG. 4 is a schematic illustrating example flow paths of an example cell expansion system, like the cell expansion system illustrated in FIG. 1, in accordance with at least one example embodiment;

FIG. 5 is a flowchart illustrating an example method for separating target cells from a source in accordance with at least one example embodiment;

FIG. 6 is a flowchart illustrating another example method for separating target cells from a source in accordance with at least one example embodiment;

FIG. 7 is a flowchart illustrating an example method for producing chimeric antigen receptor cells using a cell expansion system, like the cell expansion system illustrated in FIG. 1, in accordance with at least one example embodiment; and

FIG. 8 is a schematic of an example counterflow containment scheme for a bioreactor, like the bioreactor illustrated in FIG. 2, in accordance with at least one example embodiment.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

Cell expansion systems are cell culturing systems used to expand and differentiate cells, including both adherent and non-adherent cell types. The present disclosure relates to cell expansion systems and processes of preparing and using the same. In at least one example embodiment, the present disclosure relates to systems and methods for producing chimeric antigen receptor (“CAR”) cells (including, for example, chimeric antigen receptor T-cells (“CAR-T”) and/or chimeric antigen receptor natural killer cells (“CAR-NK”)) and/or other like cells using cell expansion systems. Example cell expansion systems are described, for example, in U.S. Pat. No. 11,702,634 on Jul. 18, 2023 and titled EXPANDING CELLS IN A BIOREACTOR, which was filed as U.S. application Ser. No. 15/943,536 as filed Apr. 2, 2018; U.S. Pat. No. 10,577,585 as issued on Mar. 3, 2020 and titled CELL EXPANSION, which was filed as U.S. application Ser. No. 15/153,396 on May 12, 2016; U.S. Pat. No. 11,685,883 as issued on Jun. 27, 2023 and titled METHODS AND SYSTEMS FOR COATING A CELL GROWTH SURFACE, WHICH, which was filed as U.S. application Ser. No. 15/616,635 on Jun. 7, 2017; U.S. Pat. No. 12,043,823 as issued on Jul. 23, 2024 and titled CELL CAPTURE AND EXPANSION, which was filed as U.S. application Ser. No. 17/702,658 as filed Mar. 23, 2022; U.S. Pat. No. 11,999,929 as issued Jun. 4, 2023 and titled METHODS AND SYSTEMS FOR COATING A CELL GROWTH SURFACE, which was filed as U.S. application Ser. No. 16/845,686 as filed Apr. 10, 2022; U.S. application Ser. No. 17/087,571 as filed Nov. 2, 2022 and titled CELL EXPANSION, which published as U.S. Pub. No. 2021/0047602 on Feb. 18, 2021; U.S. Pat. No. 11,702,634 as issued Jul. 18, 2023 and titled EXPANDING CELLS IN A BIOREACTOR, which was filed as U.S. application Ser. No. 15/943,536 on Apr. 2, 2018; U.S. application Ser. No. 18/234,470 as filed Aug. 16, 2023 and titled METHODS FOR CELL EXPANSION, DIFFERENTIATION, AND/OR HARVESTING OF NATURAL KILLER CELLS USING HOLLOW-FIBER MEMBRANES, which published as U.S. Pub. No. 2024/0076597 on Mar. 7, 2024; and U.S. application Ser. No. 18/368,879 as filed Sep. 15, 2023 and titled EXPANDING CELLS, which published as U.S. Pub. No. 2024/0101946 on Mar. 28, 2023, the entire disclosures of which are hereby incorporated by reference.

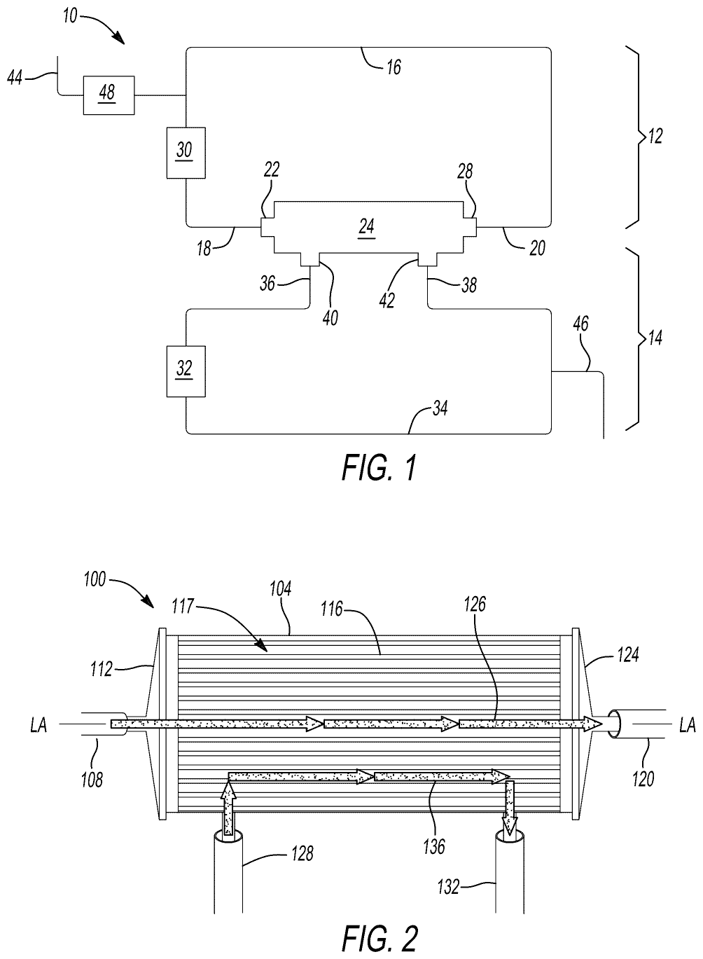

FIG. 1 is an illustration of an example cell expansion system 10. The cell expansion system 10 includes a first fluid circulation path 12 and a second fluid circulation path 14. The first fluid circulation path 12 may include, for example, a first fluid flow path 16 having opposing ends 18 and 20. The first fluid flow path 16 may be in fluid communication with a cell growth chamber 24. For example, the first opposing end 18 of the first fluid flow path 16 may be in fluid communication with a first inlet 22 of the cell growth chamber 24, and the second opposing end 20 may be in fluid communication with first outlet 28 of the cell growth chamber 24. The cell growth chamber 24 may include, or be configured to receive, a bioreactor (which may also be referred to as a hollow fiber membrane (HFM)) 117 (see FIG. 2).

The bioreactor 117 may be a standard bioreactor or a small bioreactor. The standard and small bioreactors may be similarly configured and received by the same cell expansion system 10. The standard and small bioreactors, however, have different general dimensions. A standard bioreactor may be generally selected to accommodate larger cell seeds (e.g., greater than 50 M cells) and/or to produce larger cell harvests (e.g., greater than 2 B cells), while a small bioreactor may be generally selected to accommodate smaller cell seeds (e.g., less than or equal to 50 M Cells) and/or to product smaller cell harvests (e.g., less than 3 B cells) and/or to maintain lower costs. In each instance, fluid in the first circulation path 12 may flow through an interior of a plurality of hollow fibers 116 of the bioreactor 117. In at least one example embodiment, a first fluid flow control device 30 may be operably coupled to the first fluid flow path 16 and may control the flow of fluid in first fluid circulation path 12.

The second fluid circulation path 14 may include, for example, a second fluid flow path 34 and a second fluid flow control device 32. Like the first fluid flow path 16, the second fluid flow path 34 may have opposing ends 36 and 38. The opposing ends 36 and 38 of second fluid flow path 34 may in fluid communication with an inlet port 40 and an outlet port 42 of the cell growth chamber 24. For example, a first opposing end 36 of the second fluid flow path 34 may be in fluid communication with the inlet port 40 of the cell growth chamber 24, and the second opposing end 38 of the second fluid flow path 34 may be in fluid communication with the outlet port 42. Fluid in the second circulation path 14 may be in contact with an outside of the bioreactor 117 disposed in the cell growth chamber 24. In at least one example embodiment, a second fluid flow control device 32 may be operably coupled to the second fluid flow path 34 and may control the flow of fluid in the second fluid circulation path 14.

The first and second fluid circulation paths 12, 14 may be maintained in the cell growth chamber 24 by way of the bioreactor 117, where fluid in first fluid circulation path 12 flows through an intracapillary (IC) space of the bioreactor 117 and fluid in the second circulation path 14 flows through the extracapillary (EC) space of the cell growth chamber 24. The first circulation path 12 may be referred to as the “intracapillary loop” or “IC loop”. The second fluid circulation path 14 may be referred to as the “extracapillary loop” or “EC loop”. Fluid in first fluid circulation path 12 may flow in either a co-current or counter-current direction with respect to a fluid flow in second fluid circulation path 14.

In at least one example embodiment, a fluid inlet path 44 may be fluidly associated with the first fluid circulation path 12, and a fluid outlet path 46 may be fluidly associated with the second fluid circulation path 14. The fluid inlet path 44 may permit fluid into first fluid circulation path 12, while the fluid outlet path 46 may permit fluid to exit the cell expansion system 10. In at least one example embodiment, as illustrated, a third fluid flow control device 48 may be operably associated with the fluid inlet path 44. Although not illustrated, it should be recognized that, in various other example embodiments, a fourth fluid flow control device may alternatively or additionally be associated operably associated with the first outlet path 46. In at least one example embodiment, the fluid flow control devices (including the first fluid flow control device 30 and/or the second fluid flow control device 32 and/or the third fluid flow control device 48 and/or the fourth fluid flow control device) may include a pump, valve, clamp, or any combination thereof. For example, multiple pumps, valves, and clamps can be arranged in any combination. In at least one example embodiment, the fluid flow control device may be, or include, a peristaltic pump. Fluid circulation paths (including the first fluid circulation path 12 and/or the second fluid circulation path 14) and/or inlet ports (including the fluid inlet port 44) and/or the outlet port (including the fluid outlet port 46) may include any known tubing material, and any kind of fluid—including, for example, buffers, protein containing fluid, and cell-containing fluid—can flow through the various circulation paths (including the first fluid circulation path 12 and/or the second fluid circulation path 14) and/or the inlet paths (including the fluid inlet port 44), and outlet paths (including the fluid outlet port 46). It should be recognized that the terms “fluid,” “media,” and “fluid media” are used interchangeably.

An example cell growth chamber 100 is illustrated in FIG. 2. The cell growth chamber 100 may be used as the cell growth chamber 24 of the cell expansion system 10 illustrated in FIG. 1. The cell growth chamber 100 may have a longitudinal axis (represented by the line LA-LA) and may include a cell growth chamber housing 104. The cell growth chamber housing 104 may have four openings or ports, including, for example, an intracapillary inlet port 108, an intracapillary outlet port 120, an extracapillary inlet port 128, and an extracapillary outlet port 132. A first fluid (which can also be referred to as an intracapillary fluid or media) in a first circulation path (like the first fluid circulation path 12) can enter the cell growth chamber 100 through the intracapillary inlet port 108 at a first fluid manifold end 112 of the cell growth chamber 100 and into and through the intracapillary spaces of a plurality of hollow fibers 116 and out of cell growth chamber 100 through intracapillary outlet port 120, which is located at a second fluid manifold end 124 of the cell growth chamber 100. The fluid path between the intracapillary inlet port 108 and the intracapillary outlet port 120 may define an intracapillary portion 126 of the cell growth chamber 100. A second fluid (which can also be referred to as an extracapillary media or fluid) in a second circulation path (like the second fluid circulation path 14) can enter the cell growth chamber 100 through the extracapillary inlet port 128. This second fluid contacts the extracapillary space or outside of the bioreactor 117 and exits the cell growth chamber 100 via the extracapillary outlet port 132. The fluid path between the extracapillary inlet port 128 and the extracapillary outlet port 132 may define an extracapillary portion 136 of the cell growth chamber 100.

As the second fluid comes into contact with the outside of the hollow fibers 116 small molecules (e.g., ions, water, oxygen, lactate, etc.) may diffuse through the hollow fibers 116 from the interior or intracapillary space of the hollow fibers 116 to the exterior or extracapillary space, or alternatively, or additionally, from the extracapillary space to the intracapillary space. Large molecular weight molecules (e.g., growth factors and/or proteins) are often too large to pass through the membrane walls of the hollow fibers 116 and remain in the intracapillary space (or alternatively, or additionally, in the extracapillary space) of the hollow fibers 116. The mediums defining the first and second fluids may be replaced as needed and may alternatively, or additionally, be circulated through an oxygenator and/or gas transfer module to exchange gasses, as needed. As discussed below, cells for expansion (e.g., natural killer cells and/or T-cells) may be contained within the first fluid circulation path 12 and/or the second fluid circulation path 14 and may enter the cell growth chamber 100 on one or both of the intracapillary space or the extracapillary space.

Often, cells for expansion (including, for example, natural killer cells and/or T-cells) are seeded (for example, for expansion, differentiation, and/or harvesting of cord blood derived CD34+ hematopoietic stem/progenitor cells, monocytes, macrophages, hepatocytes, and/or endothelial cells) in the intracapillary space 130, while a cell culture medium is pumped through the extracapillary space 110 to deliver nutrients to the cells via hollow-fiber membrane perfusion during expansion. However, in other variations, cells for expansion (including, for example, natural killer cells and/or T-cells) can be seeded (for example, for expansion, differentiation, and/or harvesting of cord blood derived CD34+ hematopoietic stem/progenitor cells, monocytes, macrophages, hepatocytes, and/or endothelial cells) in the extracapillary space 110, while the cell culture medium is pumped through the intracapillary space 130 to deliver nutrients to the cells via hollow-fiber membrane perfusion during expansion. In still further variations, cells for expansion (including, for example, natural killer cells and/or T-cells) may be seeded (for example, for expansion, differentiation, and/or harvesting of cord blood derived CD34+ hematopoietic stem/progenitor cells, monocytes, macrophages, hepatocytes, and/or endothelial cells) in the intracapillary space 130, while the cell culture medium is pumped through both the extracapillary space 110 and the intracapillary space 130. In such instances, movement of the cell culture medium through the intracapillary space 130 can help to remove excess cells not adhered to surfaces of the hollow-fiber membrane 101. In each instance, the material used to form the hollow-fiber membrane 101 may be any biocompatible polymeric material that is capable of being made into the hollow fibers 121. For example, synthetic polysulfone-based materials (e.g., polyethersulfones (PES)) are often used to form the hollow fibers.

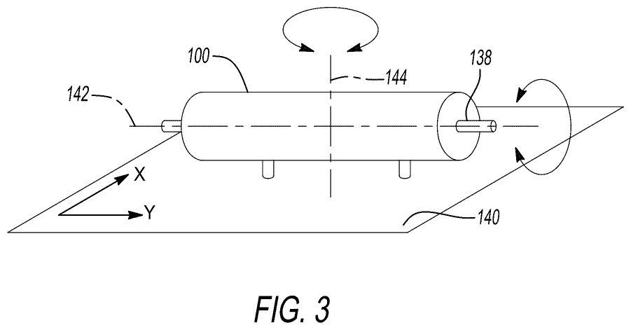

In at least one example embodiment, the cell expansion system 10 may also include a device that is configured to move or “rock” the cell growth chamber 100 relative to other components of the cell expansion system 10. The device may be a rotational and/or lateral rocking device. For example, as illustrated in FIG. 3, the cell growth chamber 100 may be rotationally connected to one or more rotational rocking components 138 and to a lateral rocking component 140. A first rotational rocking component 138 may be rotationally associated with the cell growth chamber 100. For example, the first rotational rocking component 138 may be configured to rotate the cell growth chamber 100 around a first or central rotational axis 142. In at least one example embodiment, the cell growth chamber 100 may be rotated in alternating fashion, including, for example, in a first clockwise direction and then in a second counterclockwise direction around the central axis 142.

Although not illustrated, it should be recognized that in at least one example embodiment, a second rotational rocking component may be configured to move the cell growth chamber 100 about a second rotational axis 144 that passes through a center point of the cell growth chamber 100 normal to the central axis 142. In at least one example embodiment, the cell growth chamber 100 may be rotated in alternating fashion, including, for example, in a first clockwise direction and then in a second counterclockwise direction around the second axis 144. In at least one example embodiment, the cell growth chamber 100 may also be rotated around the second axis 144 and positioned in a horizontal or vertical orientation relative to gravity. The lateral rocking component 140 may be laterally associated with the cell growth chamber 100. For example, a plane of the lateral rocking component 140 may move laterally in the x-direction and y-direction.

The rotational and/or lateral movement of the cell growth chamber 100 may reduce the settling of cells and the likelihood of cells becoming trapped within a portion of the bioreactor 117 disposed in the cell growth chamber 100. In at least one example embodiment, the rate of cells settling in the cell growth chamber 100 may be proportional to the density difference between the cells and the suspension media, according to Stoke's Law. In at least one example embodiment, a 180-degree rotation (fast) with a pause (having, for example, a total combined time of 30 seconds) repeated as described above may help to keep non-adherent cells (for example, T-cells) suspended. A minimum rotation of about 180-degrees may be preferred, however various degrees of rotation, including up to or greater than 360-degrees, may be used. Different rocking components may be used separately or may be combined in any combination. For example, a rocking component that rotates cell growth chamber 100 around central axis 142 may be combined with the rocking component that rotates cell growth chamber 100 around axis 144. Likewise, clockwise and counterclockwise rotation around different axes may be performed independently in any combination.

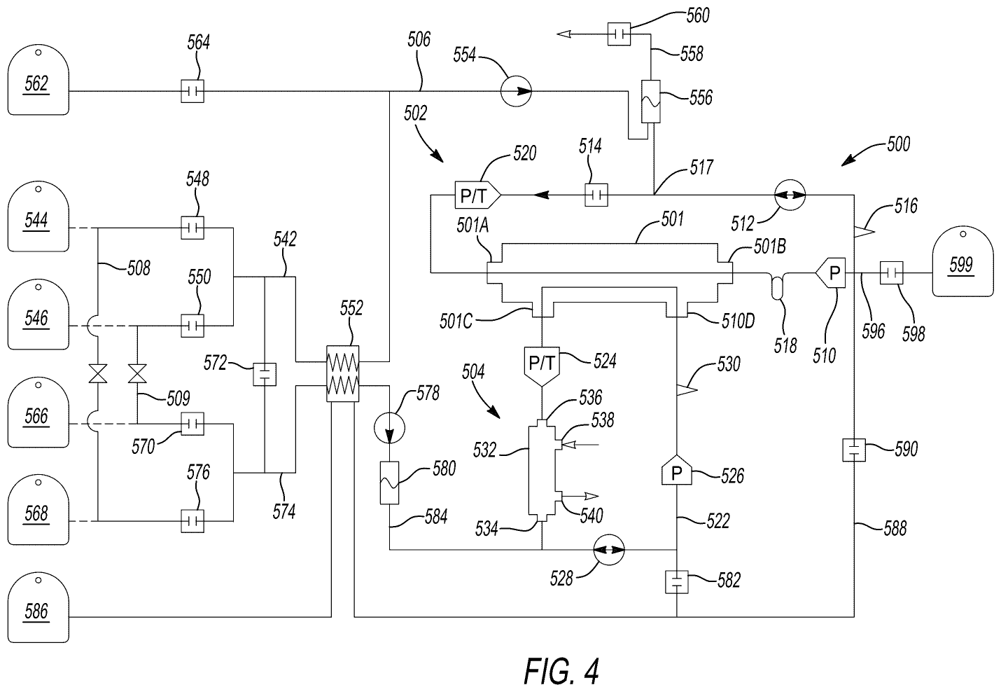

FIG. 4 is a schematic of an example cell expansion system 500, which may be like the cell expansion system 100 illustrated in FIG. 1, that illustrates example flow paths. In at least one example embodiment, the cells may be positioned in the intracapillary space, while a cell culture medium may be pumped through the extracapillary space to deliver nutrients to the cells via hollow fiber membrane perfusion during expansion. It should be recognized, however, in at least one other example embodiment, cells can be positioned in the extracapillary space, while the cell culture medium may be pumped through the intracapillary space to deliver nutrients to the cells via hollow fiber membrane perfusion during expansion. In at least one other example embodiment, cells may be positioned in the intracapillary space, while the cell culture medium may be pumped through both the extracapillary space and the intracapillary space.

As illustrated, the cell expansion system 500 may include a first fluid circulation path 502 (also referred to as the “intracapillary loop” or “IC loop”) and a second fluid circulation path 504 (also referred to as the “extracapillary loop” or “EC loop”). The first fluid flow path 506 may be fluidly associated with a cell growth chamber 501 to form first fluid circulation path 502. The cell growth chamber 501 may be used as the cell growth chamber 24 illustrated in FIG. 1 and/or the cell growth chamber 100 illustrated in FIG. 2. A first fluid may flow into cell growth chamber 501 through an intracapillary inlet port 501A. The first fluid may exit the cell growth chamber via an intracapillary outlet port 501B. In at least one example embodiment, the first fluid circulation path 502 may include a pressure gauge 510 configured to measure a pressure of the first fluid leaving the cell growth chamber 501. In at least one example embodiment, the first fluid circulation path 502 may include an intracapillary circulation pump 512 configured to control a first fluid flow rate. For example, the intracapillary circulation pump 512 may be configured to pump the first fluid in a first direction or a second direction that is opposite to the first direction. In the later instance, the intracapillary outlet port 501B may be used as an inlet, and the intracapillary inlet port 501A as an outlet. In at least one example embodiment, the first fluid circulation path 502 may include a sample port 516 and/or sample coil 518 configured for first fluid sample extraction. In at least one example embodiment, the first fluid circulation path 502 may include a pressure/temperature gauge 520 configured to detect the pressure and/or temperature of the first fluid during operation. In at least one example embodiment, the first fluid may enter the intracapillary loop 502 via valve 514. In at least one example embodiment, a portion of the cells may be flushed from the intracapillary loop 502 into a harvest bag 599, for example, via valve 598. It should be recognized that, in at least one other example embodiment, the first fluid circulation path 502 may include additional or fewer valves, pressure gauges, pressure sensors, temperature sensors, ports, and/or other devices disposed to isolate and/or measure characteristics of the first fluid along portions of the intracapillary loop 502.

A second fluid may flow into cell growth chamber 501 through an extracapillary inlet port 501C. The second fluid may leave the cell growth chamber 501 via an extracapillary outlet port 501D. In at least one example embodiment, the second fluid in the extracapillary loop 504 may contact an exterior facing surface of hollow fibers disposed in the cell growth chamber 501 thereby allowing diffusion of small molecules into and out of the hollow fibers. In at least one example embodiment, the extracapillary loop 504 may include a pressure/temperature gauge 524 configured to measure a pressure and/or temperature of the second fluid before the second fluid enters the cell growth chamber 501. In at least one example embodiment, the extracapillary loop 504 may include a pressure gauge 526 that is configured to measure a pressure of the second fluid, for example, as it leaves the cell growth chamber 501. In at least one example embodiment, the extracapillary loop 504 may include a sample port 530 configured for second fluid sample extraction.

In at least one example embodiment, the extracapillary loop 504 may include an extracapillary circulation pump 528 and an oxygenator or gas transfer module 532. For example, after leaving the cell growth chamber 501, the second fluid may pass through the extracapillary circulation pump 528 and to and through the oxygenator or gas transfer module 532. In at least one example embodiment, the extracapillary circulation pump 528 may be configured to control a second fluid flow rate. For example, like the intracapillary circulation pump 512, the extracapillary circulation pump 528 may be configured to pump the second fluid in a first direction or a second direction that is opposite to the first direction. In the later instance, the extracapillary outlet port 501D may be used as inlet, and the extracapillary inlet port 501C as an outlet.

In at least one example embodiment, the second fluid flow path 522 may be fluidly associated with the oxygenator or gas transfer module 532 via an oxygenator inlet port 534 and an oxygenator outlet port 536. For example, the second fluid may flow into the oxygenator or gas transfer module 532 via the oxygenator inlet port 534 and may leave or exit the oxygenator or gas transfer module 532 via the oxygenator outlet port 536. In at least one example embodiment, the oxygenator or gas transfer module 532 may be configured to add oxygen to and/or remove bubbles from the second fluid. For example, air and/or gas may flow into the oxygenator or gas transfer module 532 via a first filter 538 and may leave or exit (i.e., flow out of) the oxygenator or gas transfer device 532 through a second filter 540. The first and second filters 538, 540 may be configured to reduce or prevent contaminants from entering the oxygenator or gas transfer module 532. The second fluid in the second fluid circulation path 504 may be in equilibrium with gas entering the oxygenator or gas transfer module 532. In at least one example embodiment, air and/or gas may be purged from the cell expansion system 500, for example, during a priming sequence, air and/or gas may be vented to the atmosphere via the oxygenator or gas transfer module 532. It should be recognized that, in at least one other example embodiment, a second fluid circulation path 504 may include additional or fewer valves, pressure gauges, pressure sensors, temperature sensors, ports, and/or other devices disposed to isolate and/or measure characteristics of the second fluid along portions of the extracapillary loop 504.

In at least one example embodiment, an air removal chamber (ARC) 556 may be fluidly associated with the first circulation path 502. The air removal chamber 556 may include one or more ultrasonic sensors. For example, the air removal chamber 556 may include upper sensor and/or lower sensor which are configured to detect air and/or a lack of fluid and/or gas-fluid interface at certain measuring positions within the air removal chamber 556. The upper sensor may be disposed near a first end (e.g., top) of the air removal chamber 556. The lower sensor may be disposed near a second end (e.g., bottom) of the air removal chamber 556. Although ultrasonic sensors are discussed, it should be appreciated that the air removal chamber 556 may include, additionally, or alternatively, one or more other sensors, including, for example, optical sensors. Air and/or gas purged from the cell expansion system 500 during portions of a priming sequence and/or other protocols may vent to the atmosphere out air valve 560 via line 558 that may be fluidly associated with air removal chamber 556.

In at least one example embodiment, the first fluid may include cells (for example, from a first fluid container (which can also be referred to as a first media bag or a first bag) 562 and also fluid media (e.g., intracapillary media or fluid) from a second fluid container (which can also be referred to as a second media bag or a second bag) 546. Materials (i.e., cells and/or intracapillary media) form the first and second fluid containers 562, 546 may enter the first fluid circulation path 502 via a first fluid flow path 506. The first fluid container 562 may be fluidly associated with the first fluid flow path 506 and the first fluid circulation path 502 via valve 564. In at least one example embodiment, the second fluid container 546 and a third fluid container (which can also be referred to as a third media bag or third bag) 544 may be fluidly associated with the first fluid inlet path 542, for example, via valves 548 and 550, respectively, or with a second fluid inlet path 574, for example, via valves 570 and 576, respectively. In at least one example embodiment, the materials from the second fluid container 546 and/or the third fluid container 544 may be in fluid communication with a first sterile sealable input priming path 508 and/or a second sterile sealable input priming path 509.

In at least one example embodiment, a fourth fluid container (which can also be referred to as a fourth media bag or a fourth bag) 568 may include an extracapillary media, and a fifth fluid container (which can also be referred to a fifth media bag or a fifth bag) 566 may include a wash solution. Materials (i.e., extracapillary media and/or wash solution) from the fourth and fifth fluid containers 568, 566 may enter the first fluid circulation path 502 and/or the second fluid circulation path 504. For example, in at least one example embodiment, the fifth fluid container 566 may be fluidly associated with valve 570, where valve 570 is fluidly associated with first fluid circulation path 502, for example, via a distribution valve 572 and a first fluid inlet path 542. In at least one example embodiment, the fifth fluid container 566 may be fluidly associated with the second fluid circulation path 504 via the second fluid inlet path 574 and an extracapillary inlet path 584, for example, by opening valve 570 and closing distribution valve 572. The fourth fluid container 568 may be fluidly associated with valve 576, where valve 576 is fluidly associated with first fluid circulation path 502, for example, via the first fluid inlet path 542 and the distribution valve 572. In at least one example embodiment, the fourth fluid container 568 may be fluidly associated with the second fluid inlet path 574 by opening valve 576 and closing the distribution valve 572. In at least one example embodiment, the first fluid inlet path 542 and/or the second fluid inlet path 574 may be fluidly associated with an optional heat exchanger 552.

In at least one example embodiment, fluid may be advanced to the intracapillary loop 502 from the first fluid inlet path 542 and/or the second fluid inlet path 574 via an intracapillary inlet pump 554, and fluid may be advanced to the extracapillary loop 504 via an extracapillary inlet pump 578. In at least one example embodiment, an air detector 580 may also be associated with the extracapillary inlet path 584. The air detector 580 may include, for example, an ultrasonic sensor. In at least one example embodiment, the first and second fluid circulation paths 502, 504 may be fluidly associated with a waste line 588. For example, when valve 590 is in an open state or position, the intracapillary media may flow through the waste line 588 to a waste bag (also referred to as an outlet bag) 586. When valve 582 is opened, extracapillary media may flow through the waste line 588 to the waste bag 586. In at least one example embodiment, cells may be harvested, for example, via a cell harvest path 596. For example, cells from the cell growth chamber 501 may be harvested by pumping the intracapillary media containing the cells through the cell harvest path 596 and also valve 598 to a cell harvest bag 599.

In at least one example embodiment, as illustrated, the fluid in the first fluid circulation path 502 and second fluid circulation path 504 flows through cell growth chamber 501 in the same direction (i.e., a co-current configuration). Although not illustrated, it should be recognized that, in various other example embodiments, the cell expansion system 500 may also be configured to flow in a counter-current conformation. As illustrated in FIG. 4, fluid in the first fluid circulation path 502 may enter the cell growth chamber 501 at the intracapillary inlet port 501A and may leave or exit the cell growth chamber 501 at the intracapillary outlet port 501B. In at least one example embodiment, the first fluid flow path 506 may be fluidly connected to the first fluid circulation path 502, for example, via connection 517. Connection 517 may be a point or location from which the fluid may flow in opposite directions, for example, based on the direction and flow rates of the intracapillary inlet pump 554 and fluid circulation pump 512. Connection 517 may include any type of fitting, coupling, fusion, pathway, and/or tubing that allows the first fluid flow path 506 to be fluidly associated with the first fluid circulation path 502. In at least one example embodiment, connection 517 may include a T-fitting or coupling and/or a Y-fitting or coupling.

In at least one example embodiment, one or more of the gauges (e.g., pressure gauge 510 and/or pressure/temperature gauge 520 and/or pressure/temperature gauge 524 and/or pressure gauge 526), one or more of the valves (e.g., valve 514 and/or valves 548 and/or valves 550 and/or valve 560 and/or valve 564 and/or valve 570 and/or valve 572 and/or valve 576 and/or valve 582 and/or valve 590 and/or valve 596 and/or valve 598), one or more of the ports (e.g., intracapillary inlet port 501A and/or intracapillary outlet port 501B and/or extracapillary inlet port 501C and/or extracapillary outlet port 501D and/or sample port 516 and/or sample port 530 and/or oxygenator inlet port 534 and/or an oxygenator outlet port 536), one or more of the pumps (e.g., intracapillary circulation pump 512 and/or extracapillary circulation pump 528 and/or intracapillary inlet pump 554 and/or extracapillary inlet pump 578), one or more of the filters (e.g., first filter 538 and/or second filter 540), one or more coils (e.g., sample coil 518), one or more modules (e.g., oxygenator or gas transfer module 532), and/or one or more other components of the cell expansion system 500 may be in electrical communication with a control system (not shown). The control system may include a plurality of nodes, which can include various hardware, firmware, and/or software configured to control and/or communicate with the mechanical, electromechanical, and electrical components of the cell expansion system 500, including for example, a controller and a memory.

The controller (which can also be referred to as a processor), can be of any type of microcontroller, microprocessor, Field Programmable Gate Array (FPGA), Application Specific Integrated Circuit (ASIC), etc. An example controller may be the NK10DN512VOK10 microcontroller, made and sold by N9P USA, Incorporated, which is a microcontroller unit with a 32-bit architecture. Other examples controllers may include, for example, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM926EJ-S™ processors, other industry-equivalent processors, and may perform computational functions using any known or future-developed standard, instruction set, libraries, and/or architecture. The memory can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller.

In at least one example embodiment, protocols for expansion of suspension cells (like natural killer cells and/or T-cells) using cell expansion systems (like the cell expansion system 10 illustrated in FIG. 1) may implement counterflow (for example, using the intracapillary circulation pump 512 and the intracapillary inlet pump 554 as illustrated in FIG. 4) into an intracapillary space (or alternatively, or additionally, counter flow into the extracapillary space may be implemented using the extracapillary circulation pump 528 and extracapillary inlet pump 578) of a bioreactor (for example, within the first circulation path 12 of the bioreactor 24 as illustrated in FIG. 1 and/or the intracapillary portion 126 of the bioreactor 100 as illustrated in FIG. 2 and/or the first fluid circulation path 502 of the bioreactor 501 as illustrated in FIG. 4) to confine cells within the hollow fibers (or a desired region thereof) of the bioreactor (for example, such as the plurality of hollow fibers 116 disposed within or defining the bioreactor 24 as illustrated in FIGS. 1 and 2). As discussed above, large molecular weight molecules (such as from the culture media and/or the expanding cell population) are often too large to pass through the membrane walls of the hollow fibers and remain in the intracapillary space (or alternatively, or additionally, in the extracapillary space) of the hollow fibers. Small particulates such as cellular debris and viral particles likewise are too large to pass through membrane walls of the hollow fibers. The confinement of the large molecular weight molecules (which are herein referred to as “solutes”) and small particulates can often lead or progressive accumulation of various proteins, cytokines, and other waste products within the intracapillary space (or alternatively, or additionally, in the extracapillary space) of the hollow fibers, which can unfavorably impact cell viability, proliferation, and phenotype.

In various aspects, the present disclosure provides systems and methods for separating non-cellular materials and target cells from various sources prior to the material entering (or introducing the material to) a bioreactor and/or during cell expansion (or other processing) using the bioreactor (for example, such as the bioreactor 24 as illustrated in FIG. 1 and/or the bioreactor 100 as illustrated in FIG. 2 and/or the bioreactor 501 as illustrated in FIG. 4). There are several applications where removing non-cellular matter is advantageous or required. For example, certain cytokines may be necessary for the expansion of cells but may become toxic at high concentrations (e.g., IL-15 and/or IL-21) for natural killer cells; expanding T-cells may require stimulating a T-cell receptor (TCR), which is often achieved through the addition of anti-CD3 and anti-CD28 antibodies conjugated to beads, and it is often advantageous to remove the T-cell receptor stimulant during the expansion phase; and/or chimeric antigen receptor cells, including live viruses, may need to be added to the bioreactor for cellular transduction, but then removed during expansion.

In at least one example embodiment, an intracapillary loop of a cell expansion system (for example, such as the first circulation path 12 illustrated in FIG. 1 and/or the first fluid circulation path 502 illustrated in FIG. 4), or alternatively, or additionally, an extracapillary loop of a cell expansion system (for example, such as the second fluid circulation path 14 illustrated in FIG. 1 and/or the second fluid circulation path 504 illustrated in FIG. 4), may further include one or more bypass loops. The intracapillary loop and/or the extracapillary loop may include one or more valves that are configured to allow fluid or media to flow through the one or more bypass loops (for example at 20 or 38 of FIG. 1 for intracapillary loop or extracapillary loop integration, respectively). Each of the one or more bypass loops may include one or more size exclusion filters that are configured to retain certain larger materials or cells while allowing smaller materials or cells (e.g., cytokines, viruses, or other small molecules) to pass through. In at least one example embodiment, the one or more bypass loops may direct the and directing the smaller materials or cells to a waste bag. Once the smaller materials or cells are separated, flow in the intracapillary loop and/or the extracapillary loop may be reversed, such that the larger materials or cells are pushed back into the bioreactor. In at least one example embodiment, the filters may have an average pore size greater than or equal to about 3 micrometers. In at least one example embodiment, the filters may have an average pore size less than or equal to about 5 micrometers. In at least one example embodiment, the filters may have an average pore size greater than or equal to about 3 micrometers to less than or equal to about 5 micrometers.

In various aspects, the present disclosure provides systems and methods for separating non-target cells and target cells (which may also be referred to as pre-selecting target cells) from various sources prior the material entering (or introducing the material to) a bioreactor and/or during cell expansion (or other processing) using the bioreactor (for example, such as the bioreactor 24 as illustrated in FIG. 1 and/or the bioreactor 100 as illustrated in FIG. 2 and/or the bioreactor 501 as illustrated in FIG. 4). Selection of specific cell types for expansion (or other processing) often presents opportunities for pathogen contamination. Providing a separation means, and more specifically, an automated separation means, in line with the cell expansion system (like the cell expansion system 10 as illustrated in FIG. 1 and disposed between an inlet bag and an intracapillary loop) can help to maintain the sterility of the separation.

Methods for isolating or separating target cells (e.g., natural killer cells and/or T-cells) from a source may include positioning a magnetic column between a source provided, for example, along a cell inlet path (for example, the fluid inlet path 506 illustrated in FIG. 4) prior to joining, for example, the first circulation path 502 illustrated in FIG. 4. For example, in at least one example embodiment, the magnetic column may be disposed between the first fluid container 562 and valve 564 of the cell expansion system 500 illustrated in FIG. 4. In each instance, the magnetic column may include a housing that includes ferromagnetic spheres, where the ferromagnetic spheres create a torturous path for cells to migrate through and are magnetized when placed in a magnetic field.

Methods for isolating or separating target cells (e.g., natural killer cells and/or T-cells) from a source may include negative selection elimination methods. Negative election elimination methods may help to keep the target cell type free of antibody-mediated inhibition or activation, which may be observed in the instance of positive selection processes. In at least one example embodiment, methods for isolating or separating target cells (e.g., natural killer cells and/or T-cells) from a source may include using magnetically conjugated antibodies. For example, negative selection of T-cells may include magnetically separating non-target cells using anti-CD14 antibodies, anti-CD16 antibodies, anti-CD19 antibodies, anti-CD36 antibodies, anti-CD5 antibodies 6, anti-CD123 antibodies, anti-CD235 antibodies, or any combination thereof.

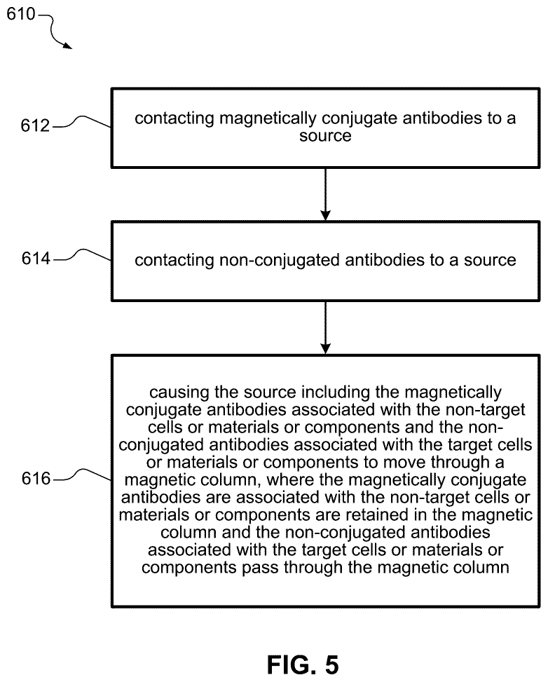

FIG. 5 illustrates an example method 610 for isolating or separating target cells from a source using magnetically conjugated antibodies and non-conjugated antibodies. The method 610 may include contacting 612 the source to the magnetically conjugate antibodies and contacting 614 the source to the non-conjugated antibodies (which may also be referred to as stimulating antibodies). The magnetically conjugated antibodies are selected to bind or associate with non-target cells or materials or components, and the non-conjugated antibodies are selected to bind or associate with the target cells and the contacting 612, 614 may occur for at least a selected time period. The non-conjugated antibodies may interact with target cells by stimulating activating receptors to enhance cell activation and/or expansion. In at least one example embodiment, the non-conjugated antibodies may include soluble antibodies. In at least one example embodiment, the non-conjugated antibodies may be associated in a non-magnetic complex.

Although illustrated as separate steps, it should be appreciated that, in various example embodiments, the magnetically conjugate antibodies and the non-conjugated antibodies may be contacted 612, 614 to the source concurrently or subsequently. For example, in at least one example embodiment, the magnetically conjugate antibodies may be contacted 612 to the source (or the source contacted 612 to the magnetically conjugated antibodies) and afterwards the non-conjugated antibodies may be contacted 614 to the source (or the source contacted 614 to the non-conjugated antibodies). In other example embodiments, the non-conjugated antibodies may be contacted 614 to the source (or the source contacted 614 to the non-conjugated antibodies) and afterwards the magnetically conjugate antibodies may be contacted 612 to the source (or the source contacted 612 to the magnetically conjugated antibodies). In still another example embodiment, the magnetically conjugate antibodies may be contacted 612 to the source (or the source contacted 612 to the magnetically conjugated antibodies) and the non-conjugated antibodies may be contacted 614 to the source (or the source contacted 614 to the non-conjugated antibodies) at the same time. For example, in at least one example embodiment, the contacting 612 of the magnetically conjugate antibodies to the source (or the contacting 612 of the source to the magnetically conjugate antibodies) and the contacting 614 of the non-conjugated antibodies to the source (or the contacting 614 of the source to the non-conjugated antibodies) may include moving the source (including non-target cells or materials or components and the target cells) from a first bag (e.g., an inlet bag) to another bag (e.g., stimulation and selection bag), where the another bag includes the magnetically conjugate antibodies and the non-conjugated antibodies.

After the magnetically conjugated antibodies are associated with the non-target cells and the non-conjugated antibodies are associated with the target cells, the method 610 may further include causing 616 the source including the magnetically conjugate antibodies and the non-conjugated antibodies to move through a magnetic column, where the magnetic column that is configured to retain the magnetically conjugate antibodies associated with the non-target cells or materials or components while allowing the non-conjugated antibodies associated with the target cells to pass therethrough and onto the intracapillary loop (for example, such as the first circulation path 12 illustrated in FIG. 1 and/or the first fluid circulation path 502 illustrated in FIG. 4), or alternatively, or additionally, to the extracapillary loop (for example, such as the second fluid circulation path 14 illustrated in FIG. 1 and/or the second fluid circulation path 504 illustrated in FIG. 4).

Methods for isolating or separating target cells (e.g., natural killer cells and/or T-cells) from a source may include using magnetic selection beads in a positive selection process. The magnetic selection beads may include, for example, magnetic beads selected to conjugated to antibodies that bind natural killer cells or T-cells, such as, for example, anti-CD56 antibodies, anti-CD3 antibodies, anti-CD28 antibodies, or any combination thereof. In some instances, binding of antibodies to target cells may induce cell activation. In at least one example embodiment, the magnetic selection beads may be contacted to the source between, for example, a first bag (e.g., an inlet bag) and the bioreactor, where the target cells associate (or form conjugates) with the magnetic bead-conjugated selection and/or stimulation antibodies. To release these target cells into the bioreactor, the methods may include removing or releasing the magnetic field from around the column and flushing the cells into the bioreactor using the movement of fluid.

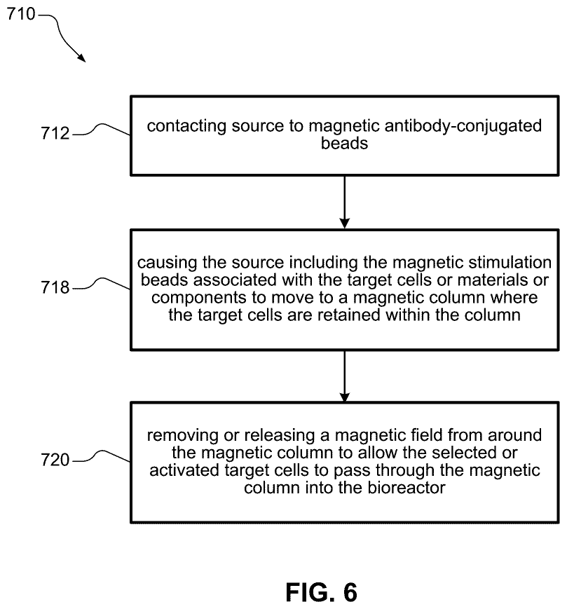

FIG. 6 illustrates an example method 710 for isolating or separating target cells from a source using magnetic selection beads. The method 710 may include contacting 712 the source to magnetic selection beads. Although not illustrated, it should be appreciated that, in various example embodiments, the method 710 may include identifying or selecting the magnetic selection beads for conjugation to selection and/or activation antibodies that bind to the target cells (e.g., natural killer cells and/or T-cells). In at least one example embodiment, the contacting 712 may include moving the source from a first bag (e.g., an inlet bag) to another bag (e.g., stimulation and selection bag), where the another bag includes the magnetic selection beads. The contacting 712 may occur for at least a selected time period.

After the magnetic stimulation beads are associated with the target cells, the method 710 may further include causing 718 the source including the magnetic stimulation beads to move to a magnetic column, where the magnetic column is configured to retain the stimulation beads associated with the target cells or materials or components. After the source including the magnetic stimulation beads are moved to the magnetic column, the method 710 may further include removing or releasing a magnetic field from around the magnetic column to allow the magnetic antibody-conjugated beads associated with the target cells to pass therethrough and onto, the intracapillary loop (for example, such as the first circulation path 12 illustrated in FIG. 1 and/or the first fluid circulation path 502 illustrated in FIG. 4), or alternatively, or additionally, to the extracapillary loop (for example, such as the second fluid circulation path 14 illustrated in FIG. 1 and/or the second fluid circulation path 504 illustrated in FIG. 4).

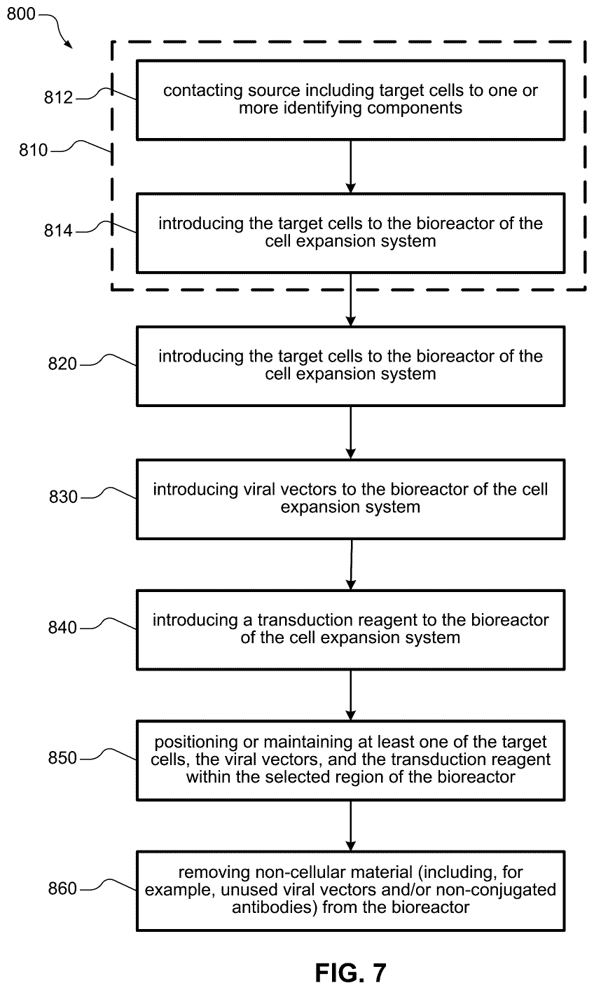

In various aspects, the present disclosure provides systems and methods for producing chimeric antigen receptor cells, or like cells, using a cell expansion system, like the cell expansion system 10 illustrated in FIG. 1 and/or the cell expansion system 500 illustrated in FIG. 4. For example, FIG. 7 illustrates an example method 800 using a cell expansion system.

The method 800 may include introducing (at step 820) target cells (e.g., natural killer cells and/or T-cells) to a bioreactor (like the bioreactor 24 as illustrated in FIG. 1 and/or the bioreactor 100 as illustrated in FIG. 2 and/or the bioreactor 501 as illustrated in FIG. 4) of the cell expansion system. In at least one example embodiment, introducing 820 the target cells may include causing the target cells to move (e.g., flow) into the bioreactor of the cell expansion system. In at least one example embodiment, introducing 820 the target cells may include placing a (first) source or inlet bag or container including the target cells in fluid communication with the bioreactor of the cell expansion system. In at least one example embodiment, introducing 820 the target cells may include placing the (first) source or inlet bag or container including the target cells in fluid communication with the cell expansion system. In at least one example embodiment, introducing 820 the target cells may include moving (manually or automatically) one or more valves from a closed position to an open position for a selected or predetermined period of time and/or removing one or more clamps.

The method 800 may include isolating or separating 810 the target cells (e.g., natural killer cells and/or T-cells) from a source. In at least one example embodiment, the target cells may be isolated or separated from a source using magnetically conjugated antibodies and non-conjugated antibodies as illustrated in FIG. 5. In at least one example embodiment, the target cells may be separated from a source using magnetic selection beads such as illustrated in FIG. 6. For example, in each instance, the method 800 may including contacting 812 the source with one or more identifying components and causing 814 the source including the one or more identifying components to move through a separating column (e.g., magnetic column), where the target cells are allowed to proceed therethrough to the bioreactor and the non-target cells are captured and/or otherwise directed, for example, to a waste bag. In at least one example embodiment, the contacting 812 of the source with the one or more identifying components may include moving the source from the (first) source or inlet bag or container to a selection bag including the one or more identifying components. The contacting 812 of the source with the one or more identifying components may occur for a selected or predetermined time period. The flow rate of the source including the one or more identifying components as moving through the separating column may be adjusted to tune separation and isolation.

The method 800 may include introducing (at step 830) viral vectors (which may also be referred to as chimeric antigen receptor-encoding retrovirus or lentivirus vectors) to the bioreactor of the cell expansion system. In at least one example embodiment, introducing 830 the viral vectors may include causing the virus to move (e.g., flow) into the bioreactor of the cell expansion system. In at least one example embodiment, introducing 830 the viral vectors may include placing a (second) source or inlet bag or container including the viral vectors in fluid communication with the bioreactor of the cell expansion system. In at least one example embodiment, introducing 830 the viral vectors may include placing the (second) source or inlet bag or container including the viral vectors in fluid communication with the cell expansion system. In at least one example embodiment, introducing 830 the viral vectors may include moving (manually or automatically) one or more valves from a closed position to an open position for a selected or predetermined period of time and/or removing one or more clamps. In at least one example embodiment, introducing 830 the viral vectors may include introducing an amount of the viral vectors such that a ratio of the target cells to the viral vectors is greater than or equal to about 0.1 to less than or equal to about 50.

The method 800 may include introducing 840 a transduction reagent to the bioreactor of the cell expansion system. In at least example embodiment, introducing 840 the transduction reagent may include causing the transduction reagent to move (e.g., flow) into the bioreactor of the cell expansion system. In at least one example embodiment, introducing 840 the transduction reagent may include placing a (third) source or inlet bag or container including the transduction reagent in fluid communication with the bioreactor of the cell expansion system. In at least one example embodiment, introducing 840 the transduction reagent may include placing the (third) source or inlet bag or container including the transduction reagent in fluid communication with the cell expansion system. In at least one example embodiment, introducing 840 the transduction reagent may include moving (manually or automatically) one or more valves from a closed position to an open position for a selected or predetermined period of time and/or removing one or more clamps. In at least one example embodiment, introducing 840 the transduction reagent may include coating one or more surface of the bioreactor with the transduction reagent. For example, one or more surfaces defining the portion of the intracapillary loop within the bioreactor may be coated with the transduction reagent. The transduction reagent coating may be a continuous or a discontinuous coating. In at least one example embodiment, the transduction reagent may coat greater than or equal to about 50% (e.g., greater than or equal to about 55%, greater than or equal to about 60%, greater than or equal to about 65%, greater than or equal to about 70%, greater than or equal to about 75%, greater than or equal to about 80%, greater than or equal to about 85%, greater than or equal to about 90%, or greater than or equal to about 95%) of the one or more surfaces. In each instance, the transduction reagent is selected to hold the target cells and the viral vectors in close proximity to encourage viral transduction. For example, VLA4/VLA5 of target cells may interact with the transduction reagent, while the transduction reagent binds with the H-domain of the viral vectors. In at least one example embodiment, the transduction reagent may include retronectin.

Although the introduction 820 of the target cells is illustrated as occurring before the introduction 830 of the viral vectors, it should be appreciated that, in various example embodiments, the viral vectors 830 may be introduced before the introduction 820 of the target cells and/or at the same time as the introduction 820 of the target cells. Although the introduction 840 of the transduction reagent is illustrated as occurring after the introduction 820 of the target cells and after the introduction 830 of the viral vectors, it should be appreciated that, in various example embodiments, the transduction reagent may be introduced before the introduction 820 of the target cells and/or before the introduction 830 of the viral vectors and/or at the same time as the introduction 820 of the target cells and/or the introduction 830 of the viral vectors. In at least one example embodiment, the transduction reagent may be introduced 840 in solution with, or as a suspension with, the target cells and/or the viral vectors.

The method 800 may include positioning or maintaining 850 at least one of the target cells, the viral vectors, and the transduction reagent within the bioreactor (and more specifically, for example, the intracapillary portion or space, or alternatively, or additionally, the extracapillary portion or space) using a counterflow containment scheme.

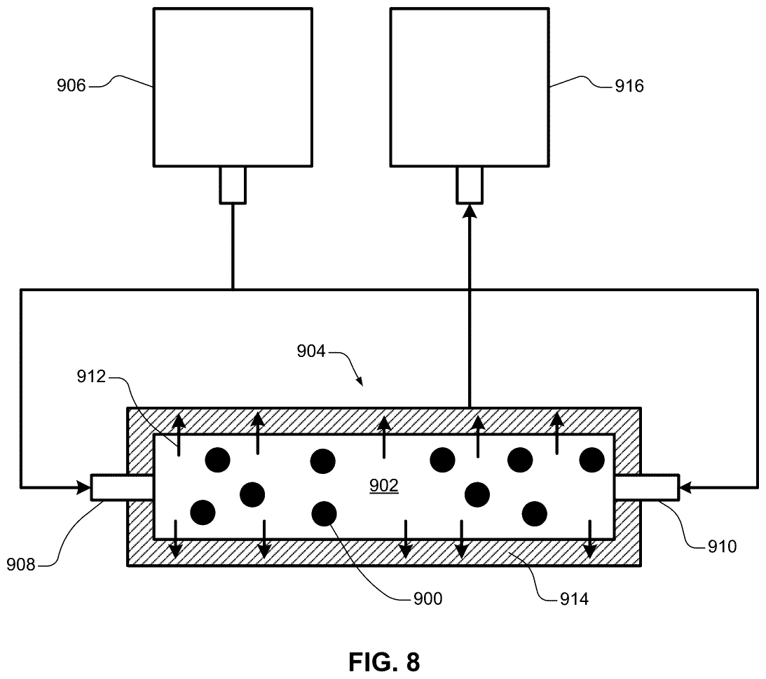

By way of example, FIG. 8 provides a schematic of an example counterflow containment scheme. As illustrated, cells and/or other materials 900 may be positioned (or repositioned) within an intracapillary portion or space 902 of a bioreactor 904. More specifically, as media or fluid may move from a media bag or container 906 it may be split between an intracapillary inlet pump (not shown) and an intracapillary circulation pump (not shown) such that the media or fluid moves into the intracapillary portion or space 902 of the bioreactor 904 through both an intracapillary inlet 908 and an intracapillary outlet 910. In contrast, the cells and/or other material 900 may be seeded and/or recirculated using unidirectional flow from the media bag or container 906 to the intracapillary portion or space 902 of the bioreactor 904 via the intracapillary inlet 908.

Counter flows may be adjusted throughout the confinement period such that cells and/or other materials might be positioned or held at selected locations or area within the bioreactor 904 between inlet and outlet locations. In at least one example embodiment, during a feeding stage, the intracapillary inlet flow rate may be about 2 times of the intracapillary circulation pump rate. For example, the intracapillary inlet flow rate may be greater than or equal to about 0.02 mL/min and the intracapillary circulation pump rate may be greater than or equal to about 0.01 mL/min. Arrows 912 illustrate the fluid movement through pores of the bioreactor 904 from the intracapillary portion or space 902 to the extracapillary portion or space 914 during the counterflow containment. A waste bag 916 may be in fluid communication with the extracapillary portion or space 914 allowing appropriate movement from the extracapillary portion 914 to the waste bag 916.

Although the cells and/or other materials 900 are illustrated in FIG. 8 as being seeded within the intracapillary portion or space 902, it should be appreciated that, in various other example embodiments, the cells and/or other materials 900 may instead be seeded instead in the extracapillary portion or space 914. In such instances counterflow containment may include moving media or fluid from the media bag or container 906 to an extracapillary inlet (not shown) and also an extracapillary outlet (not shown).

The method 810 may include removing 860 non-cellular material (e.g., unused lentivirus and/or non-conjugated antibodies). In at least one example embodiment, the removing 860 of the non-cellular material may include causing or moving the suspension or solution including the cells (e.g., natural killer cells and/or T-cells) and other materials to move (for example, pushing) into a bypass loop including one or more size exclusion filters, where the bypass loop is in communication with the intracapillary loop of a cell expansion system (for example, such as the first circulation path 12 illustrated in FIG. 1 and/or the first fluid circulation path 502 illustrated in FIG. 4), or alternatively, or additionally, the extracapillary loop of a cell expansion system (for example, such as the second fluid circulation path 14 illustrated in FIG. 1 and/or the second fluid circulation path 504 illustrated in FIG. 4). The bypass loop includes one or more size exclusion filters selected to retain cells (e.g., natural killer cells and/or T-cells), while smaller materials (e.g., antibodies, beads, and viruses) pass through and into, for example, a waste bag. In at least one example embodiment, the removing 860 of the non-cellular material may include (for example, after the causing or moving of the suspension or solution into the bypass loop) reversing flow or movement and causing or moving a clean media move through the bypass loop so as to push the cells away from the filter and back into the bioreactor (for example, for further expansion).

Although single steps are illustrated, it should be appreciated that, in various example embodiments, the method 800 may include one or more of each of the detailed steps.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for preparing chimeric antigen receptor cells using a cell expansion system, the method comprising:
contacting target cells and viral vectors within a bioreactor of the cell expansion system.

2. The method of claim 1, wherein the method further includes:
introducing the target cells to the bioreactor.

3. The method of claim 2, wherein the method further includes:
isolating the target cells from a source, the source including the target cells and a non-target material.

4. The method of claim 3, wherein the isolation of the target cells from the source includes:
contacting the source to one or more identifying components, and
causing the source including the one or more identifying components to move through a separating column, the separating column disposed in the cell expansion system in line with the bioreactor and configured to allow passage of the target cells and to either hold the non-target material or to direct the non-target material to a holding container of the cell expansion system.

5. The method of claim 4, wherein
the one or more identifying components includes:
magnetically conjugated antibodies selected to associate with the non-target material, and
non-conjugated antibodies selected to associate with the target cells; and
the separating column includes a magnetic column.

6. The method of claim 5, wherein the method further includes at least one of:
identifying the magnetically conjugated antibodies, and
identifying the non-conjugated antibodies.

7. The method of claim 4, wherein
the one or more identifying components includes:
stimulation beads selected to associate with the target material,
magnetic antibody-conjugated beads selected to associate with the target cells, or
a combination thereof; and
the separating column includes a magnetic column.

8. The method of claim 7, wherein the method further includes at least one of:
identifying the stimulation beads, and
identifying the magnetic antibody-conjugated beads.

9. The method of claim 2, wherein the method further includes:
introducing the viral vectors to the bioreactor.

10. The method of claim 1, wherein the method further includes:
contacting the target cells and viral vectors with a transduction reagent within the bioreactor.

11. The method of claim 10, wherein the method further includes:
introducing the transduction reagent to the bioreactor.

12. The method of claim 11, wherein the introduction of the transduction reagent includes:
coating on one or more surfaces of the bioreactor with the transduction reagent.

13. The method of claim 12, wherein the introduction of the transduction reagent includes:
placing the transduction reagent in suspension with the target cells and the viral vectors.

14. The method of claim 10, wherein the contacting includes:
maintaining the target cells, the viral vectors, and the transduction reagent within the bioreactor using counterflow containment.

15. The method of claim 10, wherein the method further includes:
removing non-cellular material from the bioreactor, the non-cellular material including unused viral vectors and unused identifying components.

16. The method of claim 15, wherein the removal of the non-cellular material from the bioreactor includes:
causing material in the bioreactor to flow through a bypass loop including one or more size exclusion filters.

17. The method of claim 16, wherein the removal of the non-cellular material from the bioreactor further includes:
reversing flow; and
moving a clean media through the bypass loop.

18. A method for preparing chimeric antigen receptor cells using a cell expansion system, the method comprising:
introducing target cells to a bioreactor of the cell expansion system, the introduction of the target cells including isolating the target cells from a source including the target cells and a non-target material, the isolation of the target cells from the source including:
contacting the source to one or more identifying components, and
causing the source including the one or more identifying components to move through a separating column, the separating column disposed in the cell expansion system in line with the bioreactor and configured to allow passage of the target cells and to either hold the non-target material or to direct the non-target material to a holding container of the cell expansion system;
introducing viral vectors to the bioreactor to associate with the target cells;
introducing a transduction reagent to the bioreactor to associate with the target cells and the transduction reagent; and
removing non-cellular material from the bioreactor by causing material in the bioreactor to flow through a bypass loop including one or more size exclusion filters, the non-cellular material including unused viral vectors and unused identifying components.

19. The method of claim 18, wherein the method includes
maintaining the target cells, the viral vectors, and the transduction reagent within the bioreactor using counterflow containment.

20. The method of claim 18, wherein the introduction of the transduction reagent includes:
coating on one or more surfaces of the bioreactor with the transduction reagent.
